(19) United States
(12) Patent Application Publication
(10) Pub. No.: US 2006/0136032 A1
(43) Pub. Date: Jun. 22, 2006

(54) Balloon catheter having a balloon with hybrid porosity sublayers

(75) Inventors: Nora V. Legarda, San Jose, CA (US); Edwin Wang, Tustin, CA (US); Avegel Hernando, Union City, CA (US)

Correspondence Address: FULWIDER PATTON, 6060 CENTER DRIVE, 10TH FLOOR, LOS ANGELES, CA, 90045, US

(73) Assignee: ADVANCED CARDIOVASCULAR SYSTEMS, INC.

(21) Appl. No.: 11/015,716

(22) Filed: Dec. 16, 2004

(51) Int. Cl. A61F 2/06

(52) U.S. Cl. 623/1.11; 604/103.06

(57) ABSTRACT

formed of at least two sublayers of the porous polymeric material which have a different porosity. Additionally, in one embodiment, the sublayers of porous polymeric material have other characteristics which vary, such as tensile strength and orientation. As a result, the balloon of the invention has an improved combination of characteristics such as a low profile with a desired compliance and rupture pressure.

This invention relates generally to catheters, and particularly intravascular catheters for use in percutaneous transluminal coronary angioplasty (PTCA) or for the delivery of stents.

In percutaneous transluminal coronary angioplasty (PTCA) procedures a guiding catheter is advanced in the patient's vasculature until the distal tip of the guiding catheter is seated in the ostium of a desired coronary artery. A guidewire is first advanced out of the distal end of the guiding catheter into the patient's coronary artery until the distal end of the guidewire crosses a lesion to be dilated. A dilatation catheter, having an inflatable balloon on the distal portion thereof, is advanced into the patient's coronary anatomy over the previously introduced guidewire until the balloon of the dilatation catheter is properly positioned across the lesion. Once properly positioned, the dilatation balloon is inflated with inflation fluid one or more times to a predetermined size at relatively high pressures so that the stenosis is compressed against the arterial wall and the wall expanded to open up the vascular passageway. Generally, the inflated diameter of the balloon is approximately the same diameter as the native diameter of the body lumen being dilated so as to complete the dilatation but not overexpand the artery wall. After the balloon is finally deflated, blood flow resumes through the dilated artery and the dilatation catheter and the guidewire can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate of angioplasty alone and to strengthen the dilated area, physicians now normally implant an intravascular prosthesis, generally called a stent, inside the artery at the site of the lesion. Stents may also be used to repair vessels having an intimal flap or dissection or to generally strengthen a weakened section of a vessel or to maintain its patency. Stents are usually delivered to a desired location within a coronary artery in a contracted condition on a balloon of a catheter which is similar in many respects to a balloon angioplasty catheter, and expanded within the patient's artery to a larger diameter by expansion of the balloon. The balloon is deflated to remove the catheter and the stent left in place within the artery at the site of the dilated lesion. See for example, U.S. Pat. No. 5,507,768 (Lau et al.) and U.S. Pat. No. 5,458,615 (Klemm et al.), which are incorporated herein by reference.

An essential step in effectively performing a PTCA procedure is properly positioning the balloon catheter at a desired location within the coronary artery. To properly position the balloon at the stenosed region, the catheter shaft must be able to transmit force along the length of the catheter shaft to allow it to be pushed through the vasculature. However, the catheter shaft must also retain sufficient flexibility to allow it to track over a guidewire through the often tortuous vasculature. Additionally, the catheter must have good crossability (i.e., the ability of the catheter distal end to cross stenosed portions of the vascular anatomy).

Accordingly, it would be a significant advance to provide a catheter with an improved combination of characteristics such as compliance, rupture pressure and profile for improved performance. This invention satisfies these and other needs.

### SUMMARY OF THE INVENTION

The invention is directed to a catheter with a balloon having a porous polymeric material layer formed of at least two sublayers of the porous polymeric material which have a different porosity. Additionally, in one embodiment, the sublayers of porous polymeric material have other characteristics which vary, such as tensile strength and orientation. As a result, the balloon of the invention has an improved combination of characteristics such as a low profile with a desired compliance and rupture pressure.

The catheter generally comprises an elongated shaft having an inflation lumen and a guidewire lumen, and a balloon on a distal shaft section with a proximal end section and a distal end section secured to the shaft so that an interior chamber of the balloon is in fluid communication with the inflation lumen. The balloon typically has a nonporous layer in addition to the porous polymeric layer, making the balloon fluid-tight, so that the balloon inflates by retaining inflation fluid within the interior chamber of the balloon. Although discussed herein in terms of a presently preferred embodiment in which the porous polymeric layer is an outer layer relative to the nonporous layer, it should be understood that alternatively the porous polymeric layer can be an inner layer. In a presently preferred embodiment, the porous polymeric layer is impregnated, along at least a section thereof, with a polymeric material which at least partially fills the pores of the porous polymeric material. In one embodiment, the nonporous layer is omitted and the porous polymeric layer is sufficiently impregnated with a polymeric material to reduce the fluid-permeability of the porous polymeric material so that the balloon is inflatable.

A variety of suitable porous polymers may be used to form the porous polymeric layer of the balloon, including expanded polytetrafluoroethylene (ePTFE), an ultra high molecular weight polyolefin such as ultra high molecular weight polyethylene (UHMWPE), porous polyethylene, porous polypropylene, and porous polyurethane. In a presently preferred embodiment, the porous polymeric material has a node and fibril microstructure. For example, ePTFE and UHMWPE (also known as expanded UHMWPE), typically has a node and fibril microstructure comprising nodes interconnected by fibrils.

The different porosity sublayers are formed of the same porous polymeric material (e.g., ePTFE). Thus, the sublayers readily fuse or otherwise bond together, to form a single porous polymeric layer of a single porous material having a hybrid porosity which varies along the radial direction (i.e., from the inner surface toward the outer surface of the porous polymeric layer).

The porous polymeric layer is formed of at least one sublayer of a first porosity and at least one sublayer of a second porosity higher than the first porosity. However, it typically has two or more sublayers of the first porosity and two or more sublayers of the second porosity. In one embodiment, the first porosity is about 60% to about 65%, and the second porosity is about 70% to about 80%. However, a variety of suitable porosities may be used depending on the porous polymeric material used and the desired balloon performance, including porosities ranging from about 40% to about 95%, more specifically about 55% to about 85%. The first porosity is typically at least about 10 porosity percentage points different than the second porosity (e.g., a first porosity of about 65% and a second porosity of about 75% or more). As discussed in more detail below, in a presently preferred embodiment, the second (i.e., higher) porosity sublayer is an outer sublayer relative to the first porosity sublayer, although in alternative embodiments it is an inner sublayer relative to the first porosity sublayer.

The porosity of the porous polymeric material affects the compressibility and resulting stiffness of the sublayer formed of the porous polymeric material. The sublayers with the higher porosity are softer and more compressible, providing for improved low profile and stent retention. Specifically, the balloon can be compressed a greater amount to a smaller outer diameter during manufacture of the balloon catheter, to form a low profile configuration for advancement within a patient's body lumen. In one embodiment, the higher porosity sublayers are the outer-most layers of the balloon. As a result, in an embodiment having a stent mounted on the balloon for delivery and deployment within a patient's body lumen, the stent is radially pressed into the outer, high porosity sublayers during stent mounting, providing improved stent retention on the balloon. Moreover, in an embodiment having a therapeutic agent such as a drug delivery coating on a surface of the stent, the high compressibility of the higher porosity outer sublayers prevents or inhibits damage to the drug delivery coating which can otherwise occur during mounting of the stent onto a balloon having stiffer outer layers.

In one embodiment, the sublayers of different porosity also have a different tensile strength. Additionally, the sublayers have a different node and fibril microstructure (i.e., a different average node height to width ratio).

The porous polymeric sublayers typically comprise helically wound material heat fused together into a tubular shape to form the balloon porous sublayers. In one embodiment, the sublayers of different porosity have a different helical winding angle. The winding angle affects the orientation of the node and fibril microstructure of the polymer in the resulting balloon layer, and consequently, the compliance of the balloon (i.e., the degree of expansion resulting from a given increase in inflation pressure, expressed as millimeters of expansion per atmosphere of inflation pressure).

The balloon porous layer is formed of a variety of sublayers of differing porosity in order to form a balloon with an improved balance of the often competing considerations of profile, compliance, and rupture pressure. In contrast to a balloon formed of sublayers of porous material with the same porosity, bulk density, and matrix tensile strength, the balloon of the invention has an improved combination of characteristics due to the different sublayers of porous polymeric material used to make the porous polymeric layer. The balloon has a low profile due to the increased compressibility provided by the high porosity sublayers. Moreover, in the embodiment having the higher porosity sublayers as the outer-most sublayers, the balloon has improved stent retention and stent drug delivery coating integrity. These and other advantages of the invention will become more apparent from the following detailed description and exemplary drawings.

### BRIEF DESCRIPTION OF THE DRAWINGS

FIG. 1 is an elevational view partially in section of a balloon catheter embodying features of the invention.

FIGS. 2-3 are transverse cross sectional views of the balloon catheter of FIG. 1, taken along lines 2-2, and 3-3, respectively.

FIG. 4 is an enlarged longitudinal cross sectional partial view of the balloon of FIG. 1.

FIG. 5 is a transverse cross sectional views of the balloon of FIG. 4, taken along line 5-5.

FIG. 6 illustrates a sheet of porous polymeric material being helically wrapped on a mandrel during formation of the porous polymeric layer of the balloon.

### DETAILED DESCRIPTION OF THE PREFERRED EMBODIMENTS

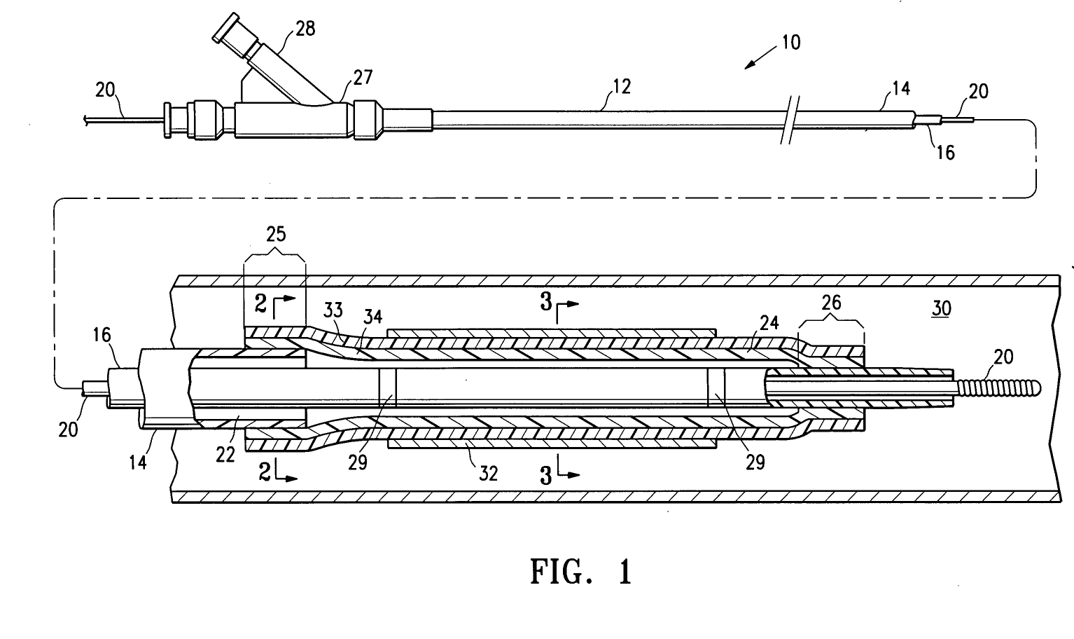

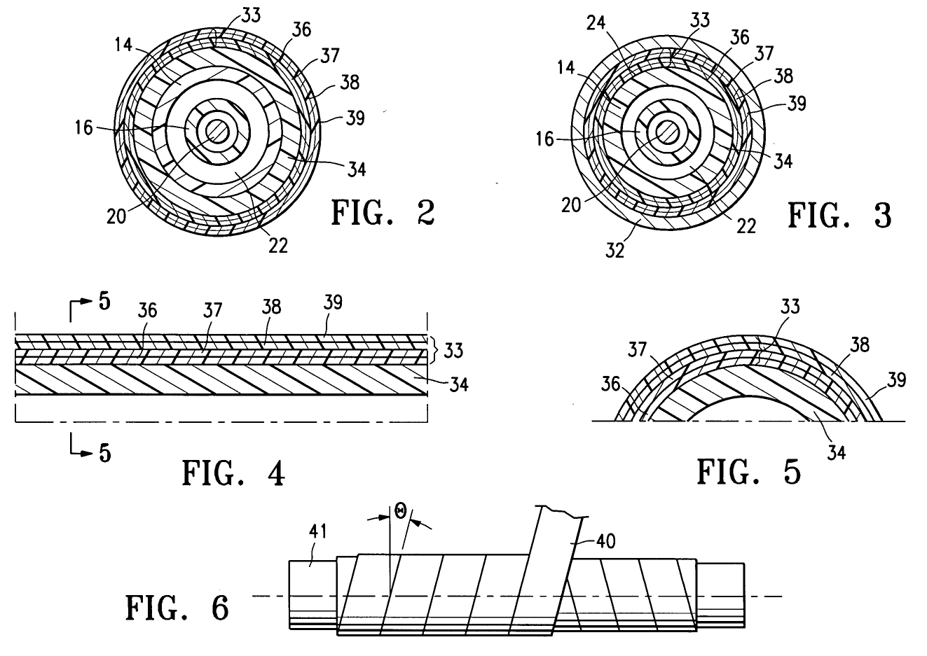

FIG. 1 illustrates an over-the-wire type balloon catheter 10 embodying features of the invention. Catheter 10 generally comprises an elongated catheter shaft 12 and an inflatable balloon 24 on a distal shaft section. In the illustrated embodiment, the shaft comprises an outer tubular member 14 defining an inflation lumen 22 therein, and an inner tubular member 16 defining a guidewire lumen 18 therein configured to slidingly receive a guidewire 20. Specifically, in the illustrated embodiment, the coaxial relationship between outer tubular member 14 and inner tubular member 16 defines annular inflation lumen 22, as best shown in FIG. 2 illustrating a transverse cross section of the distal end of the catheter shown in FIG. 1, taken along line 2-2. In the embodiment illustrated in FIG. 1, the guidewire lumen 18 extends to the proximal end of the catheter. Inflatable balloon 24 has a proximal skirt section 25 sealingly secured to the distal end of outer tubular member 14 and a distal skirt section 26 sealingly secured to the distal end of inner tubular member 16, so that the balloon interior chamber is in fluid communication with inflation lumen 22. Radiopaque markers 29 on the inner tubular member 16 facilitate viewing the location of the balloon. An adapter 27 at the proximal end of catheter shaft 12 is configured to provide access to guidewire lumen 18, and to direct inflation fluid through arm 28 into inflation lumen 22.

The balloon 24 is illustrated in FIG. 1 in a noninflated configuration prior to complete inflation thereof. In the embodiment of FIG. 1, balloon 24 has an essentially wingless noninflated configuration. However, in alternative embodiments (not shown), the balloon has a noninflated configuration with folded wings wrapped around the catheter. The distal end of catheter 10 may be advanced to a desired region of the patient's body lumen in a conventional manner with the balloon 24 in a deflated configuration, and the balloon 24 inflated by directing inflation fluid into the balloon interior, to perform a medical procedure such as dilatation or delivery of a stent. In the embodiment illustrated in FIG. 1, an expandable stent 32 is mounted on the working length of the balloon 24 for delivery and deployment within a patient's body lumen 30. FIG. 3 illustrates a transverse cross section of the balloon catheter of FIG. 1, taken along line 3-3.

The balloon 24 has a porous outer layer 33 and an inner layer 34 extending the full length of the balloon, from the proximal skirt section 25 to the distal skirt section 26. The inner surface of the outer layer 33 is preferably bonded to the inner layer 34, as for example by fusion bonding and/or adhesive bonding, and the balloon 24 proximal and distal skirt sections 25, 26 are bonded to the shaft 12, preferably by fusion and/or adhesive bonding. Although not illustrated a compression member such as a shape memory band, a superelastic band, a swaged band, or a coil, and preferably a swaged band, may be provided on the proximal and/or distal skirt sections 25, 26 to enhance the strength of the connection between the balloon 24 and shaft 12.

Balloon porous outer layer 33 preferably comprises a microporous polymeric material having a node and fibril microstructure such as ePTFE. Although discussed below primarily in terms of the embodiment in which the outer layer 33 is ePTFE, it should be understood that a variety of suitable materials can be used to form outer layer 33. The inner layer 34 is formed of a polymeric material preferably different from the polymeric material of the outer layer 33, and in a presently preferred embodiment is an elastomeric nonporous layer. Inner layer 34 limits or prevents leakage of inflation fluid through the microporous ePTFE to allow for inflation of the balloon 24. The inner layer 34 is preferably formed of an elastomeric material to facilitate deflation of the balloon 24 to a low profile deflated configuration, including polyurethanes, silicone rubbers, polyamide block copolymers, dienes, and the like. Inner layer 34 may consist of a separate layer which neither fills the pores nor disturbs the node and fibril structure of the ePTFE layer 33, or it may at least partially fill the pores of the ePTFE layer 33.

The ePTFE porous polymeric layer 33 comprises at least two adjacent sublayers of ePTFE porous polymeric material having a different porosity. As best illustrated in FIG. 4, showing an enlarged longitudinal cross sectional partial view of the balloon 24 of FIG. 1, in the illustrated embodiment the ePTFE porous polymeric layer 33 is formed of two inner sublayers 36, 37 and two outer sublayers 38, 39. The two inner sublayers 36, 37 are formed of the ePTFE porous polymeric material having a first porosity and the two outer sublayers 38, 39 are formed of the ePTFE porous polymeric material and having a second porosity greater than the first porosity. As a result, the outer sublayers 38, 39 are preferably softer than the inner sublayers 37, 38. In an alternative embodiment, the outer sublayers 38, 39 have a lower porosity than the inner sublayers 36, 37, so that the two outer sublayers 38, 39 are stiffer than the two inner sublayers. Although illustrated with two sublayers of each porosity, it should be understood that the number of sublayers may vary. For example, the number of sublayers of a given porosity generally varies from 1 to about 3, and the number of total sublayers making up the porous polymeric layer 33 generally varies from about 2 to about 6. Typically, sublayers of two different porosities are used to form the porous polymeric layer 33, although additional sublayers of different porosities may be provided in alternative embodiments.

Preferably, the inner sublayers 36, 37 are formed of material having a porosity (i.e., the first, lower porosity) which ranges from about 60% to about 70%, and the outer sublayers 38, 39 are formed of material having a porosity (i.e., the second, higher porosity) which ranges from about 70% to about 85%, prior to any porosity changing processing steps during balloon manufacture. The percent porosity of the sublayers may change as the result of processing steps in which the sublayers are stretched and/or compacted during manufacture of the balloon. The sublayers of different porosity are preferably subjected to the same processing steps, and are stretched and/or compacted by the same or similar amounts during the processing steps. As a result, the second porosity preferably remains higher than the first porosity in the finished balloon as part of a catheter system, and is typically about 10 to about 25 porosity percentage points higher. The outer, higher porosity sublayers 38, 39 are more compressible than the inner sublayers 36, 37, at least prior to the processing steps during balloon manufacture which stretch and/or compact the sublayers and compression of the stent 32 onto the balloon.

The outer, higher porosity sublayers 38, 39 have a tensile strength which is the same as or different than that of the two inner sublayers 36, 37. In one embodiment, the outer, higher porosity sublayers 38, 39 have a lower tensile strength than the inner sublayers 36, 37. For example, the outer, higher porosity sublayers 38, 39 have a low or medium matrix tensile strength of about 15,000 to about 35,000 psi, and the inner, lower porosity sublayers 36, 37 have a high matrix tensile strength of about 60,000 to about 70,000 psi in one embodiment.

The ePTFE layer 33 is preferably formed according to a method in which ePTFE polymeric material is wrapped with overlapping or abutting edges and then heated to fuse the wrapped material together into a tubular shape. FIG. 6 illustrates a sheet 40 of porous polymeric material being helically wrapped on a mandrel 41 during formation of the porous polymeric layer 33. The sheet 40 is helically wound in a first direction at a first angle (Ø, as measured relative to a cross sectional plane perpendicular to the longitudinal axis of the balloon), and is being helically wound in an opposite direction at the same angle (Ø). The portion wrapped in the first direction will form one sublayer (e.g., sublayer 36) and the portion wrapped in the second direction will form a second sublayer (e.g., sublayer 37). In an alternative embodiment, the sublayers are wrapped in the same direction. The helically wrapped material is heated to fuse the overlapping or abutting edges of a sublayer together and to fuse the adjacent sublayers together. Typically, all the desired sublayers are wound, one on top of the other, before being heated, so that the overlapping or abutting edges of a sublayer are heat fused together at the same time the sublayer is heat fused to adjacent sublayers. However, the sublayers can alternatively be heated before the being combined with the adjacent sublayer. The sheet 40 of polymeric material preferably has the desired microstructure (e.g., porous and/or node and fibril) before being wrapped and heated on the mandrel.

Preferably, the sublayers of the porous polymeric layer 33 are configured to provide a balloon 24 having a rated burst pressure less than the inflation pressure at which the shaft 12 will rupture. For example, in one embodiment, the rated burst pressure of the balloon 24 is less than about 25 atm. The rated burst pressure, calculated from the average rupture pressure, is the pressure to which 95% of the balloons can be pressurized without rupturing.

The tensile strength, porosity, and winding angle of the porous polymeric material all effect the rupture pressure and compliance of the resulting sublayers, and thus of the balloon formed therefrom. For example, the sheet 40 of porous polymeric material is typically stronger in one direction verses another. As a result, the compliance can be effected by the orientation of the wrapped material, which can be changed by changing the winding angle (Ø). In a presently preferred embodiment, the balloon 24 is a semi-compliant balloon, with a compliance of less than 0.045 mm/atm, and more preferably with a compliance of about 0.025 mm/atm to about 0.04 mm/atm from nominal to the rated burst pressure. Alternatively, the balloon 24 is a non-compliant balloon with a compliance of less than about 0.025 mm/atm from nominal to the rated burst pressure, or a highly compliant balloon with a compliance of greater than about 0.045 mm/atm from nominal to the rated burst pressure.

The two inner sublayers 36, 37 can be formed from a single sheet of porous polymeric material wrapped in a first direction and then back over itself in the opposite direction in the same or a different winding angle. Alternatively, the two inner sublayers 36, 37 can be formed from multiple sheets of the porous polymeric material having the same porosity, wrapped in either the same or varying angles and in either the same or opposite directions. The two outer sublayers 38, 39 can be similarly formed.

The helical winding angle (as measured in a cross sectional plane perpendicular to the longitudinal axis of the balloon) of the outer, higher porosity sublayers 38, 39 may be different than or the same as the helical winding angle of the two inner sublayers 36, 37. Generally, the winding angle is about 15 to about 35 degrees. In one embodiment, the outer, higher porosity sublayers 38, 39 have a helical winding angle, which is larger than the helical winding angle of the two inner sublayers 36, 37 (for example, the outer, higher porosity sublayers 38, 39 have a helical winding angle of about 20 to about 30 degrees, and the inner sublayers 36, 37 having a helical winding angle of about 26 to about 40 degrees). As a result, the microstructure of the ePTFE of the outer sublayers 38, 39 is preferably oriented such that the outer sublayers 38, 39 are more compliant than the inner sublayers 36, 37. The different angle is typically produced by using a sheet of porous polymeric material having a different width than the sheet used to make the inner sublayer, although a variety of suitable methods may be used including changing the degree of overlap of adjacent edges of the wrapping.

The sublayers are heated to fuse the sublayers together and form the porous layer 33. The resulting tube of ePTFE polymeric material is typically further processed by being stretched, heat treated, compacted, and heat treated again, to provide the desired properties such as the desired dimension, and dimensional stability (i.e., to minimize changes in length occurring during inflation of the balloon). The completed ePTFE layer 33 is then bonded to or otherwise combined with elastomeric liner 34 either before or after layer 34 is bonded to the shaft.

The stent 32 is mounted onto the outer surface of the balloon 24 using conventional methods, including crimping the stent to a radially compressed configuration on the balloon. The crimped stent 32 is compressed into the outer sublayers of the outer layer 33 of the balloon. For example, the outer layer 33 of the balloon typically protrudes into the spaces between adjacent struts of the crimped stent 32, providing improved stent retention. In one embodiment, stent 32 has a drug delivery coating (not shown) on the outer and/or inner surface of the stent 32.

A stent delivery balloon having a nonporous elastomeric layer and a hybrid porosity ePTFE porous polymeric layer consisting of two inner ePTFE sublayers of a first porosity of about 65% and two outer ePTFE sublayers of a second porosity of about 80% was prepared, with a nominal outer diameter of about 3.0 mm (i.e., the outer diameter at an inflation pressure about 9 atm). The balloon had the same compliance as a balloon otherwise similarly formed but having a nonhybrid porosity ePTFE layer consisting of four sublayers of ePTFE of 65% porosity. However, the balloon had a lower rupture pressure than the nonhybrid porosity ePTFE layer balloon, and specifically, a rated burst pressure of about 24 atm (compared to about 27 atm for a four-layer constant porosity balloon). The balloon had a smaller outer diameter in the low profile deflated configuration for introduction into the patient's body lumen. For example, a stent delivery balloon with two sublayers of 65% porosity and two sublayers of 80% porosity, and having a stent mounted thereon, had a crimped stent profile which was about 0.0007 to about 0.0009 inches less than the crimped stent profile of a four-layer constant porosity balloon.

The dimensions of catheter 10 are determined largely by the size of the balloon and guidewire to be employed, the catheter type, and the size of the artery or other body lumen through which the catheter must pass or the size of the stent being delivered. Typically, the outer tubular member 14 has an outer diameter of about 0.025 to about 0.04 inch (0.064 to 0.10 cm), usually about 0.037 inch (0.094 cm), and the wall thickness of the outer tubular member 14 can vary from about 0.002 to about 0.008 inch (0.0051 to 0.02 cm), typically about 0.003 to 0.005 inch (0.0076 to 0.013 cm). The inner tubular member 16 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). The overall length of the catheter 10 may range from about 100 to about 150 cm, and is typically about 143 cm. Preferably, balloon 24 has a length about 0.8 cm to about 6 cm, and an inflated working diameter of about 2 to about 10 mm.

Inner tubular member 16 and outer tubular member 14 can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives. Although the shaft is illustrated as having an inner and outer tubular member, a variety of suitable shaft configurations may be used including a dual lumen extruded shaft having a side-by-side lumens extruded therein. Similarly, although the embodiment illustrated in FIG. 1 is an over-the-wire type balloon catheter, the catheter of this invention may comprise a variety of intravascular catheters, such as a rapid exchange type balloon catheter. Rapid exchange catheters generally comprise a shaft having a relatively short guidewire lumen extending from a guidewire distal port at the catheter distal end to a guidewire proximal port spaced a relatively short distance from the distal end of the catheter and a relatively large distance from the proximal end of the catheter. Although discussed in terms of a preferred embodiment directed to a catheter balloon, it should be understood that other expandable medical devices or components thereof having a porous polymeric layer, such as vascular grafts and stent covers, may be formed according to the invention.

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed:

1. A balloon catheter, comprising:
a) an elongated shaft having an inflation lumen and a guidewire lumen; and
b) a balloon on a distal shaft section with a proximal and distal end section secured to the shaft so that an interior chamber of the balloon is in fluid communication with the inflation lumen, and having a porous polymeric material layer comprising at least two adjacent sublayers of the porous polymeric material having a different porosity and extending from the proximal end section to the distal end section of the balloon.

2. The balloon catheter of claim 1 wherein the porous polymeric layer comprises one or more sublayers formed of the porous polymeric material and having a first porosity of about 60% to about 65%, and one or more outer sublayers formed of the porous polymeric material and having a second porosity of about 70% to about 80%.

3. The balloon catheter of claim 1 wherein the porous polymeric layer comprises two or more sublayers formed of the porous polymeric material and having a first porosity, and two or more sublayers formed of the porous polymeric material and having a second porosity greater than the first porosity.

4. The balloon catheter of claim 3 wherein the second porosity sublayers are outer sublayers relative to the first porosity sublayers, so that the first porosity sublayers are inner sublayers, and so that the outer sublayers are softer than the inner sublayers.

5. The balloon catheter of claim 4 wherein the outer sublayers have a lower tensile strength than the inner sublayers.

6. The balloon catheter of claim 4 wherein the porous polymeric sublayers comprise helically wound tape heat fused together into a tubular shape, and the outer sublayers have a helical winding angle which is greater than a helical winding angle of the inner sublayers, the winding angle being measured relative to a cross sectional plane perpendicular to the longitudinal axis of the balloon.

7. The balloon catheter of claim 3 wherein the second porosity sublayers are inner sublayers relative to the first porosity sublayers, so that the first porosity sublayers are outer sublayers, and so that the outer sublayers are stiffer than the inner sublayers.

8. The balloon catheter of claim 1 wherein the adjacent sublayers are heat fusion bonded together along the entire length thereof to form the porous polymeric layer of the balloon.

9. The balloon catheter of claim 1 wherein the porous polymeric material has a node and fibril microstructure.

10. The balloon catheter of claim 1 wherein the porous polymeric material is selected from the group consisting of expanded polytetrafluoroethylene and ultrahigh molecular weight polyolefin.

11. The balloon catheter of claim 1 wherein the at least two adjacent sublayers of different porosity have a different tensile strength.

12. The balloon catheter of claim 1 wherein the balloon includes a nonporous layer on an inner or an outer surface of the porous polymeric layer.

13. The balloon catheter of claim 1 wherein the porous polymeric sublayers comprise helically wound material, heat fused together into a tubular shape.

14. The balloon catheter of claim 13 wherein the at least two adjacent sublayers of different porosity have a different helical winding angle.

15. A balloon catheter, comprising:
a) an elongated shaft having an inflation lumen and a guidewire lumen; and
b) a balloon on a distal shaft section with a proximal and distal end section secured to the shaft so that an interior chamber of the balloon is in fluid communication with the inflation lumen, and having a nonporous layer, and a porous polymeric material layer on an outer surface of the nonporous layer, the porous polymeric material layer comprising two or more inner sublayers of the porous polymeric material having a first porosity and two or more outer sublayers of the porous polymeric material having a second porosity greater than the first porosity, so that the outer sublayers are softer than the inner sublayers.

16. The balloon catheter of claim 15 wherein the catheter is a stent delivery catheter with a stent mounted on the balloon, the stent being compressed into at least an outer-most of the outer sublayers.

17. The balloon catheter of claim 16 wherein the stent carries a therapeutic agent.

18. The balloon catheter of claim 15 wherein all the sublayers extend from the proximal end section to the distal end section of the balloon.

19. The balloon catheter of claim 15 wherein the porous polymeric sublayers comprise helically wound tape heat fused together into a tubular shape, the two inner sublayers comprising helically wound material which is helically wound in a first direction at a first angle to form one of the two inner sublayers and helically wound in an opposite direction at the same angle to form a second of the two inner sublayers.

20. The balloon catheter of claim 19 wherein the two outer sublayers are helically wound at an angle different from the first angle.
